(19) United States
(12) Patent Application Publication
(10) Pub. No.: US 2004/0224324 A1
(43) Pub. Date: Nov. 11, 2004

(54) Happiar mapping

(75) Inventor: Paul H. Dear, Cambridge (GB)

Correspondence Address: PALMER & DODGE, LLP, KATHLEEN M. WILLIAMS, 111 HUNTINGTON AVENUE, BOSTON, MA, 02199, US

(73) Assignee: Medical Research Council

(21) Appl. No.: 10/740,183

(22) Filed: Dec. 18, 2003

Related U.S. Application Data
(63) Continuation of application No. PCT/GB02/02775, filed on Jun. 18, 2002.

(30) Foreign Application Priority Data
Jun. 18, 2001 (GB) 0114852.7

(51) Int. Cl. C12Q 1/68; G06F 19/00; G01N 33/48; G01N 33/50

(52) U.S. Cl. 435/6; 702/20

(57) ABSTRACT

The invention provides a method of mapping a nucleic acid, comprising the steps of: a) preparing a HAPPY mapping panel comprising a plurality of nucleic acid samples derived from a nucleic acid to be mapped; b) labelling the nucleic acids in the mapping panel; c) preparing a plurality of probes which are complementary to marker sequences present in the nucleic acid to be mapped and immobilising the probes on a solid phase; d) bringing one or more members of the mapping panel into contact with each probe, such that the probe hybridises to sequences in the mapping panel which are complementary thereto; and e) removing non-hybridised nucleic acids and detecting hybridisation of mapping panel nucleic acids to the probe.

The present invention relates to improved methods for mapping nucleic acid molecules, which allows the rapid and automated mapping of genomes. The method is based on HAPPY mapping, and is performed on molecular arrays.

HAPPY mapping is a method which has been developed for linkage mapping of the genome of any organism. It was first described using single haploid sperm as a DNA source by Dear et al. in 1989 (see Dear and Cook, (1989) NAR 17:6795) and later adapted to use multiple diploid cells as a DNA source, followed by DNA dilution and aliquotting into mapping panel members, each containing ideally 0.69 haploid equivalents, using which marker linkage can be assessed; the technique has been reviewed and employed in several publications (for example, Dear and Cook, (1993) NAR 21:13-20; Piper et al., (1998) Genome Res. 8:1299-1307; and various references cited therein).

Fundamentally, HAPPY mapping involves the breaking of genomic DNA into fragments which are physically separated to provide a panel of samples, each containing an amount of DNA preferably equal to less than one haploid equivalent of the genome in question (and ideally 0.69 haploid equivalents if sampled from bulk genomic DNA). The samples are then screened for presence of a series of markers. Markers which are located close together in the genome will cosegregate to a greater extent than markers which are more distant in the genome. By analysis of a cosegregation table obtained with a marker panel, the order and spacing of the markers in the genome can be deduced.

The main applications for HAPPY mapping include genome and gene mapping, detection of strain diversity, population analysis, epidemiology, gene expression and the demonstration of phylogenetic and taxonomic relationships.

One of the drawbacks of HAPPY mapping, and indeed any mapping procedure, is the requirement to carry out very large numbers of analyses on a large panel of samples in order to determine linkage. For example, a HAPPY mapping panel generally includes at least 96 samples, each of which has to be screened for the presence or absence of two or more markers in order to generate a linkage map. Fine resolution mapping, of course, requires the screening of the panel members with more than two markers. Mapping is thus a highly labour-intensive procedure.

Another drawback for HAPPY mapping is that, in its most common embodiment, the screening step requires for each marker a set of PCR primers to amplify the marker region from the sample nucleic acid. Whilst the use of PCR aids automation and high throughput of screening reactions, the requirement to generate at least two primers per marker, and more where nested or heminested PCR is employed, leads to additional cost and complexity.

There is thus a need in the art for a HAPPY mapping approach which reduces the complexity of PCR-based marker detection, and allows the performance of large numbers of mapping reactions in an efficient manner.

### SUMMARY OF THE INVENTION

The invention provides a reverse hybridisation HAPPY mapping procedure in which panels of probes are tested with members of a HAPPY mapping panel, as opposed to the conventional approach in which the panel members are tested with individual probes. This allows large numbers of markers to be mapped simultaneously in a HAPPY mapping panel and, potentially, the ability to map many thousands of markers in one day or less.

According to a first aspect, therefore, there is provided a method for mapping a nucleic acid, comprising the steps of:

a) preparing a HAPPY mapping panel comprising a plurality of nucleic acid samples derived from a nucleic acid to be mapped, each member of the said panel containing a sampling of DNA fragments representing an amount equal by mass to 2 or fewer coies of the nucleic acid to be mapped;

b) labelling the nucleic acids in the mapping panel;

c) preparing a plurality of probes which are complementary to marker sequences present in the nucleic acid to be mapped and immobilising the probes on a solid phase;

d) bringing one or more members of the mapping panel into contact with each of said plurality of probes, such that the probes hybridise to sequences in the mapping panel which are complementary thereto;

e) removing non-hybridised nucleic acids and detecting hybridisation of mapping panel nucleic acids to the probe.

Advantageously, the mapping panel members are pre-amplified prior to hybridisation to the probes. Advantageously, the preamplification is non-specific. Labels may be incorporated at the preamplification stage.

Each member of the mapping panel may thus be simultaneously screened with multiple probes. For example, if a multiwell plate is used for the hybridisation reactions, one probe may be immobilised in each well and nucleic acids from the mapping panel member to be tested added to each well; positive hybridisation will only take place in those wells containing a probe which is complementary to nucleic acids in the mapping panel member. Alternatively, probes may be spotted on to glass slides or membranes and incubated in a single reaction with the nucleic acids of the mapping panel member. Each member of the mapping panel is thus brought, in turn or simultaneously, into contact with each probe.

The procedure may be highly automated. For example, a large number of replicas of the plate or slide or other support comprising the probes may be made and used to screen all of the mapping panel members simultaneously. Thus, if the mapping panel comprises 96 members, 96 replicas of the probe panel may be used to screen every member for the presence or absence of every probe in a single session. The probe panels may then be read and the signals deconvoluted automatically as well, leading to a fully automated, high-throughput mapping procedure.

Unlike previously-described HAPPY mapping procedures, the procedure of the present invention as described above requires no marker-specific PCR. However, the complexity of the mapping panel members may be modulated by varying the conditions under which they are preamplified. If only a proportion of the genomic sequences in the mapping panel member are amplified, the complexity of the member will be reduced, which may in some cases be desirable to enhance the specificity of the hybridisations. Any marker will remain mappable providing that it contains at least one sequence whose counterpart remains represented in the members of the mapping panel after pre-amplification.

In certain cases, the high complexity of the nucleic acids present in the mapping panel members and/or the immobilised probes may complicate the hybridisations or reduce their selectivity. Therefore, if the probes are known (from their method of production) to contain a certain class of sequence, then the mapping panel members may advantageously be pre-amplified using PCR primers designed to preferentially amplify sequences of that class. In the extreme case, the mapping panel may be pre-amplified with a set of specific PCR primers designed, inter alia, to amplify only those exact sequences known to be present in the set of probes. In a preferred aspect of the present invention, therefore, preamplification of the mapping panel member nucleic acids is performed using primers which are designed to selectively amplify nucleic acids corresponding to sequences known or expected to be present in the probes. For instance, PCR primers may be based on the markers themselves, and used together with a proportion of random primers to enrich the amplified sequences for those which contain the markers. This procedure is advantageously performed indiscriminately in each mapping panel member, such that all sequences of the relevant class are preferentially amplified in all panel members in which they are present. The subsequent hybridisation reaction will identify which markers are present in which members of the mapping panel.

Hybridisation of the probes to primers used in the amplification reaction may be prevented by size fractionation of the panel members, to remove the primers, or by the use of non-labelled primers which will fail to be detected in the subsequent hybridisation reactions or by the use of washing conditions of a stringency sufficient to prevent hybridisation of primers, where these are substantially shorter than their amplification products.

Thus, in an advantageous embodiment the invention provides a method as described above in which, during the preamplification step, the mapping panel members are enriched for sequences present in said members which are complementary to the probes. Preferably, the mapping panel members are amplified using primers which are homologous to or complementary to the probes or to the general class of sequences represented in the probes. Complexity may also be reduced by, for example, size selection of nucleic acid fragments in the probe, selection according to restriction enzyme specificity, and the like. As an example, if the probes are cloned EcoRI fragments of >500 nucleotides in length, the mapping panel member nucleic acids may be cleaved with EcoRI and size-selected to remove material of less than about 500 nucleotides. This greatly reduces the complexity of the probe and thus complications in the reaction.

If the probes themselves are complex (and not necessarily representing a particular class of sequence; for instance, arbitrarily-chosen large cloned fragments of genomic DNA), then their complexity may be reduced prior to immobilisation by pre-amplification with one or more primers which, inter alia, amplify any subset of sequences members of which may be expected to be found in all probes. For example, if the probes are large cloned fragments of human DNA, then preamplification may be performed with primers complementary to the termini of Alu elements or other motifs common in human DNA, thereby amplifying only the subset of sequences which fortuitously lie between closely-opposed examples of such motifs. Alternatively, techniques known in the art may be applied to preferentially amplify only those portions of the probes which fortuitously lie within restriction-enzyme fragments of a defined size range.

In any case where such selective pre-amplification is used to reduce the complexity of the probes, the same or a similar method is advantageously applied uniformly to all members of the mapping panel, reducing the complexity of the mapping panel member nucleic acids whilst maintaining correpospondence between the subsets of sequences preserved in the mapping panel member nucleic acids and in the probes.

For example, the initial set of probes may consist of large (>>10 kb) cloned genomic fragments (possibly also contaminated with DNA from the host bacterium). In such a case, it may be advantageous to reduce the probe complexity before they are immobilised, for example by amplification with primers designed to amplify only an arbitrary subset of sequences (and, advantageously, sequences which are characteristic of the cloned DNA rather than of the host bacterium). In such a case, the pre-amplification of the mapping panel members would advantageously be done using similar primers, to ensure correspondence between the subsets of sequences preserved in both probes and panel.

In a further aspect, the invention relates to a method for encoding the contents of a microtitre plate so that they can be tracked though further procedures and manipulations. The method of the invention allows microtitre plate contents to be tracked even if they are transferred or combined with samples in another plate, or loaded onto a gel.

In a first embodiment of this aspect of the invention, a solid-phase marker such as inert fluorescent microspheres (e.g., Molecular Probes A3703) is added to some of the wells in the plate; the pattern of wells thus labelled represents a unique “signature” of the plate contents. For example, the position of the markers can encode a binary number.

In a preferred embodiment, microspheres are added to a microtitre plate destined for PCR; the particles do not interfere with the reaction, and are loaded onto the gel when the samples are subsequently analysed. The ‘code’ of the microtitre plate is transferred to the gel—the wells containing the fluorescent particles light up when the gel is photographed under UV light. The particles do not migrate into the gel during electrophoresis. This overcomes a general problem in tracking samples in electrophoresis —namely that it is easy to print a barcode or similar on a microtitre plate, but difficult to transfer the coding to a gel. In an alternative embodiment, DNA is loaded into some of the unused wells of the gel to encode a binary number; however, this requires the user to read the number on the microtitre plate, and then arrange for the same number to be encoded on the gel. With the system according to the inventnion, the robot which sets up the PCRs can also add the fluorescent particles, and the ‘code’ is transferred to the gel upon loading, with no further intervention or opportunity for error.

The invention is moreover applicable to further embodiments. For instance, where reactions are prepared by taking one set of reagents from one plate (e.g. a set of PCR templates) and another set from another plate (e.g. a set of PCR primers), each source-plate can be encoded by spiking certain wells with the particles. Conveniently, the coding can occupy the first row of wells in one plate, and the last row in the other plate. The reaction plate (containing the contents of the two source plates) then bears both sets of encoding markers; by imaging the plate under UV illumination the codes can be seen and verified. Alternatively, the codes will be seen when the samples are analysed by gel electrophoresis.

Multiple colours can be used to provide more complex codes, or to allow two or more codes to be superimposed in the same set of wells.

### DETAILED DESCRIPTION OF THE INVENTION

Although the general techniques mentioned herein are well known in the art, reference may be made in particular to Sambrook et al., Molecular Cloning, A Laboratory Manual (1989) and Ausubel et al., Short Protocols in Molecular Biology (1999) 4^{th} Ed, John Wiley & Sons, Inc (as well as the complete version Current Protocols in Molecular Biology).

1. Definitions

A “mapping panel” as referred to herein is a panel of nucleic acid fragments which have been separated into separate samples, or members. Each member of the panel may consist of some fraction (typically ½ or ⅓rd) of the fragmented DNA isolated from a single haploid cell, as in Dear & Cook (1989). More generally, each member may consist of a sample of fragmented DNA prepared from two or more haploid cells or from one or more diploid cells, and ideally containing an amount of DNA equal in mass to 0.69 genomes (i.e., 0.69 haploid equivalents); this amount ensures that, assuming a Poisson distribution of sequences sampled from bulk DNA, approximately half of all markers are represented in each sample; however, amounts of DNA between about 0.2 and 1 fall within the acceptable range.

The mapping panel used in the invention may be any mapping panel which contains DNA fragments derived from genomic DNA or any other source which it is intended to map. Advantageously, it comprises at least two members, and advantageously about 4, 8, 16, 32, 64, 96, 100, 110, 128, 256 or more members. The use of 96 members is convenient. Further members may be present as control samples.

A “marker” is a nucleic acid sequence which may be identified in linkage studies, for example by PCR or hybridisation analysis. Advantageously it is substantially unique within the genome under analysis such that identification thereof is unambiguous.

“Probes” are nucleic acid molecules which may be used to detect markers. In the context of the present invention, the probes are immobilised in the solid phase, and the mapping panel is in the liquid phase.

2. HAPPY mapping

General techniques for HAPPY mapping are well known in the art and have been extensively described in the literature. The following disclosures, which comprise detailed descriptions of HAPPY mapping, are incorporated herein by reference in their entirety: Konfortov et al., Genome Res. 2000 November;10(11):1737-42; Williams and Firtel, Genome Res. 2000 November;10(11):1658-9; Piper et al., Genome Res. 1998 December;8(12):1299-307; Lynch et al., Genomics. 1998 Aug. 15;52(1):17-26; Dear et al., Genomics. 1998 Mar. 1;48(2):232-41; Walter et al., Nucleic Acids Res. 1993 Sep. 25;21(19):4524-9; Dear and cook, Nucleic Acids Res. 1993 Jan 11;21(1):13-20; Dear and Cook, Nucleic Acids Res. 1989 Sep. 12;17(17):6795-807.

Markers used in HAPPY mapping may be any single-copy sequence that can be isolated by cloning, PCR amplification or other means. In conventional HAPPY mapping, markers are defined by PCR primer pairs which can be used to detect the markers by amplification in the mapping panel. This is not necessary in the method of the invention; the invention is thus not dependent on PCR, and allows markers which cannot successfully be amplified to be mapped. PCR-amplified HAPPY mapping markers may, if desired, be used in the method of the invention. Preferably, however, the markers are not identified and/or obtained by PCR amplification.

The mapping panel itself is derived either from one or more haploid cells or one or more diploid cells when mapping a genome, or from one or more representative copies of the nucleic acid it is intended to map. Broadly, each member of the mapping panel should contain randomly-chosen fragments of the genome or other nucleic acid to be mapped, such that any given sequence from the genome (or other nucleic acid) has about a 50% probability (Poisson) of being present in any particular member of the panel; if the panel is derived from the DNA of multiple haploid cells or of one or more diploid cells, then this may be achieved if each sample contains (ideally) 0.69× the mass of the [haploid] genome or other nucleic acid. Generally, DNA is handled such as to minimise unintentional strand breakage, such as by agarose encapsulation (Cook, (1984) EMBO J. 3:1837-1842, which refers to encapsulation in agarose ‘microbeads’; encapsulation in either ‘blocks’ or ‘strings’ of agarose is described in the more recent HAPPY mapping references as set forth herein). The DNA is broken into fragments of the desired size. Typically, HAPPY mapping fragments for genome mapping are of the order of 10-3000 kb in size. It will be understood that smaller fragments will increase fine resolution, but larger fragments are necessary in order to avoid gaps in the map where markers are separated by larger distances and do not show linkage over small fragment sizes. The size of mapping panel fragments may be optimised by a person skilled in the art, as desired.

The nucleic acid is then subdivided into samples, which make up the mapping panel. Again, further breakage should be avoided until the nucleic acid has been aliquoted into samples. The samples may then be preamplified using a whole genome PCR approach to amplify every sequence present in the panel, or subjected to more specific preamplification as discussed above.

3. Probe Panels

In accordance with the present invention, the mapping panel members are hybridised to sets of probes which are immobilised on to a solid surface. Preferably, the probes are immobilised in an addressable fashion. For example, they may be arrayed.

Probes may be natural or synthetic nucleic acid molecules which are advantageously present in single copy in the haploid genome equivalent used to make up the mapping panel. Preferably, probes are derived from nucleic acid to be mapped by amplification, restriction endonuclease cleavage and cloning or any other technique capable of isolating nucleic acid sequences. Advantageously, the probes are tested to ensure that they are present in single copy in the genome.

Probes may also be prepared synthetically, based on sequence information obtained from the nucleic acid to be mapped. Probes as referred to herein are composed of units which are either nucleotides or nucleotide analogues. Generally speaking, a nucleotide analogue is a compound which is capable of being incorporated in a chain of nucleotide residues and which is capable of hybridising in a base-specific manner with a base of a complementary nucleic acid chain. Analogues useful in the present invention are moreover substrates for chain-extending enzymes.

A nucleotide analogue may be a modified nucleotide wherein the base is modified, for example so as to affect base-pairing properties; and/or wherein the sugar or backbone moiety is modified, for example as in the amide linked backbones of PNA; and/or wherein the phosphate moiety is modified.

Backbone-modified nucleic acids include methylphosphonates, phosphorothioates and phosphorodithioates, where both of the non-bridging oxygens are substituted with sulphur; phosphoroamidites; alkyl phosphotriesters and boranophosphates. Achiral phosphate derivatives include 3′-O′-5′-S-phosphorothioate, 3′-S-5′-O— phosphorothioate, 3′-CH₂-5′-O— phosphonate and 3′-NH-5′-O-phosphoroamidate. Peptide nucleic acids replace the entire phosphodiester backbone with a peptide linkage.

Sugar modifications are also used to enhance stability and affinity. The α-anomer of deoxyribose may be used, where the base is inverted with respect to the natural β-anomer. The 2′-OH of the ribose sugar may be altered to form 2′-O-methyl or 2′-O-allyl sugars, which provides resistance to degradation without compromising affinity.

Modification of the heterocyclic bases preferably maintains proper base pairing. Some useful substitutions include deoxyuridine for deoxythymidine; 5-methyl-2′-deoxycytidine and 5-bromo-2′-deoxycytidine for deoxycytidine. 5-propynyl-2′-deoxyuridine and 5-propynyl-2′-deoxycytidine have been shown to increase affinity and biological activity when substituted for deoxythymidine and deoxycytidine, respectively.

Synthetic probes are preferably 7-40 residues in length overall. Usually a short probe with 8-10 residues is used, but probes with up to 20 or up to 40 residues are also useful. However, in most instances the immobilised probes are DNA fragments which are typically larger, being either PCR products (typically 50-2000 bp) or cloned DNA fragments (anything up to several hundred kb using presently available techniques).

Probes may incorporate a base analogue that promotes degenerate binding, by having the ability to base pair with two or three of the natural bases, or universal, by forming base pairs with each of the natural bases without discrimination. Such analogues may be used, in conjunction with conventional randomisation techniques, in the manufacture of the probes. However, it is preferred that the probes are highly sequence-specific in their binding and do not hybridise to degenerate sequences.

4. Immobilisation

Methods for immobilising nucleic acid probes to solid surfaces are well known in the art. For example, probe arrays are produced by immobilising pluralities of molecules of known composition to a solid phase. Typically, the molecules are immobilised onto or in discrete regions of a solid substrate. The substrate may be porous to allow immobilisation within the substrate or substantially non-porous, in which case the molecules are typically immobilised on the surface of the substrate.

The solid substrate may be made of any material to which the molecules can be bound, either directly or indirectly. Examples of suitable solid substrates include flat glass, silicon wafers, mica, ceramics and organic polymers such as plastics, including polystyrene and polymethacrylate. The surface may be configured to act as an electrode or a thermally conductive substrate (which may enhance the hybridisation or discrimination process). These may be interfaced with a permeation layer or a buffer layer. It may also be possible to use semi-permeable membranes such as nitrocellulose or nylon membranes, which are widely available. The semi-permeable membranes may be mounted on a more robust solid surface such as glass. The surfaces may optionally be coated with a layer of metal, such as gold, platinum or other transition metal. A particular example of a suitable solid substrate is the commercially available BiaCore™ chip (Pharmacia Biosensors).

Preferably, the solid substrate is generally a material having a rigid or semi-rigid surface. In preferred embodiments, at least one surface of the substrate will be substantially flat.

The solid substrate is conveniently divided up into sections. This may be achieved by techniques such as photoetching, or by the application of hydrophobic inks, for example Teflon-based inks (Cel-line, USA).

Discrete positions, in which each different molecules or groups of molecular species are located may have any convenient shape, e.g., circular, rectangular, elliptical, wedge-shaped, etc.

Attachment of a plurality of molecules to the substrate may be by covalent or non-covalent (such as electrostatic) means. The plurality of molecules may be attached to the substrate via a layer of intermediate molecules to which the plurality of molecules bind. For example, the plurality of molecules may be labelled with biotin and the substrate coated with avidin and/or streptavidin. A convenient feature of using biotinylated molecules is that the efficiency of coupling to the solid substrate can be determined easily. Since the plurality of molecules may bind only poorly to some solid substrates, it may be necessary to provide a chemical interface between the solid substrate (such as in the case of glass) and the plurality of molecules. Examples of suitable chemical interfaces include various silane linkers and polyethylene glycol spacer. Another example is the use of polylysine coated glass, the polylysine then being chemically modified if necessary using standard procedures to introduce an affinity ligand. Nucleic acids may be immobilised directly to a polylysine surface (electrostatically). Other methods for attaching molecules to the surfaces of solid substrate by the use of coupling agents are known in the art, see for example WO98/49557. The molecules may also be attached to the surface by a cleavable linker.

In one embodiment, molecules are applied to the solid substrate by spotting (such as by the use of robotic micropipetting techniques—Schena et al., 1995, Science 270: 467-470) or ink jet printing using for example robotic devices equipped with either pins or piezo electric devices as in the known art.

Polymers such as nucleic acids or polypeptides may also be synthesised in situ using photolithography and other masking techniques whereby molecules are synthesised in a step-wise manner with incorporation of monomers at particular positions being controlled by means of masking techniques and photolabile reactants. For example, U.S. Pat. No. 5,837,832 describes a method for producing DNA arrays immobilised to silicon substrates based on very large scale integration technology. In particular, U.S. Pat. No. 5,837,832 describes a strategy called “tiling” to synthesise specific sets of probes at spatially-defined locations on a substrate. U.S. Pat. No. 5,837,832 also provides references for earlier techniques that may also be used.

The size of array elements is from 0.1×0.1 microns and above as can be ink jet printed onto a patterned surface or created by photolithography or physical masking.

Immobilised molecules may also serve to bind further molecules to complete manufacture of the array. For example, nucleic acids immobilised to the solid substrate may serve to capture further nucleic acids by hybridisation, or polypeptides. Similarly, polypeptides may be incubated with other compounds, such as other polypeptides. It may be desirable to permanently “fix” these interactions using, for example UV crosslinking.

5. Screening

Sets of probes according to the invention may be screened by hybridising the immobilised probes with mapping panel sample nucleic acid. In an advantageous embodiment, all samples of the mapping panel may be screened in parallel by using replicas of the immobilised probe array. Thus, if 96 mapping panel members are prepared, all 96 may be screened in parallel using 96 probe array replicas.

Alternatively, since the probes are immobilised, probe arrays may be reused; after a hybridisation reaction, probe arrays may be washed clean of mapping panel nucleic acids, and used to screen another mapping panel member. For example, 32 replicas of the probe array may be used to screen a 96-sample mapping panel in three hybridisation experiments.

There is also the opportunity to use multicolour (or other discriminatory methods) to allow more than one panel member to be hybridised to the probe array simultaneously. For example, if one panel member is labelled with a fluorophore of one colour, and a second member with a fluorophore of a second colour, then the members can be mixed and hybridised simultaneously to the arrayed probes, as long as the two colours of fluorescence can be distinguished to deconvolute the signal, and as long as the arrayed probe is in in excess relative to the mapping panel member such that hybridisation of components of one mapping panel member does not interfere with hybridisation of components of the other. (In theory the approach can be extended to any number of mapping panel members given enough resolvable fluorophores etc; it is difficult to find large numbers of distinct conventional fluorophores, but it may be possible to use nanocrystals which have very tightly defined spectral characteristics.)

In this way, large genomes may be screened highly efficiently and rapidly.

Hybridisation conditions may be determined according to principles known to those skilled in the art. In general, it will be desired to detect only precise identity between the probes and the sample nucleic acid sequences. Thus, hybridisation is preferably carried out at high stringency.

Stringency of hybridisation refers to conditions under which polynucleic acids hybrids are stable. Such conditions are evident to those of ordinary skill in the field. As known to those of skill in the art, the stability of hybrids is reflected in the melting temperature (Tm) of the hybrid which decreases approximately 1 to 1.5° C. with every 1% decrease in sequence homology. In general, the stability of a hybrid is a function of sodium ion concentration and temperature. Typically, the hybridisation reaction is performed under conditions of high stringency.

As used herein, high stringency refers to conditions that permit hybridisation of only those nucleic acid sequences that form stable hybrids in 1 M Na+ at 65-68° C. High stringency conditions can be provided, for example, by hybridisation in an aqueous solution containing 6× SSC, 5× Denhardt's, 1% SDS (sodium dodecyl sulphate), 0.1 Na+ pyrophosphate and 0.1 mg/ml denatured salmon sperm DNA as non specific competitor. Following hybridisation, high stringency washing may be done in several steps, with a final wash (about 30 min) at the hybridisation temperature in 0.2-0.1× SSC, 0.1% SDS. It is common, when probe and/or target are likely to be rich in repeated motifs, to use as competitor repeat-enriched (or simply total genomic) DNA from the species in question, to block hybridisation between repeats. For example, hybridisations involving human DNA are often blocked using either human ‘Cot1 DNA’ (this being the fraction of bulk human DNA which rapidly re-hybridises after denaturing) or simply total human DNA (which contains an insignificant amount of any particular non-repeated sequence).

Moderate stringency refers to conditions equivalent to hybridisation in the above described solution but at about 60-62° C. In that case the final wash is performed at the hybridisation temperature in 1× SSC, 0.1% SDS.

Low stringency refers to conditions equivalent to hybridisation in the above described solution at about 50-52° C. In that case, the final wash is performed at the hybridisation temperature in 2× SSC, 0.1% SDS.

It is understood that these conditions may be adapted and duplicated using a variety of buffers, e.g. formamide-based buffers, and temperatures. Denhardt's solution and SSC are well known to those of skill in the art as are other suitable hybridisation buffers (see, e.g. Sambrook, et al., eds. (1989) Molecular Cloning: A Laboratory Manual, Cold Spring Harbor Laboratory Press, New York or Ausubel, et al., eds. (1990) Current Protocols in Molecular Biology, John Wiley & Sons, Inc.). Optimal hybridisation conditions have to be determined empirically, as the length and the GC content of the probe also play a role.

6. Uses

The improved HAPPY mapping techniques of the invention may be applied to any mapping project. Thus, mapping of genomes or genomic DNA, such as chromosomes, has already been shown to be susceptible to the application of HAPPY mapping techniques and is susceptible to the application of the improved methods described herein. Further uses of the invention may become apparent to one skilled in the art on the basis of this description.

Preferred applications for the present, or indeed any, HAPPY mapping technology are set forth below.

A. Haplotyping

As described above, the method of HAPPY mapping relies upon the random breakage and random sampling of genomic DNA to produce a set (panel) of samples containing (typically) sub-genomic amounts of DNA. Sequences (markers) which are often found together in the same members of the panel (i.e., which co-segregate) can be inferred to lie close together in the genome, compared to the average size of the fragments. Radiation hybrid mapping shares some features with HAPPY mapping, but breakage is achieved by irradiation of living ‘donor’ cells followed by fusion to unirradiated ‘host’ cells of a different species; some donor chromosome fragments are retained in the resulting hybrids, which are then analysed for their content of donor markers.

In normal use, the markers analysed by either method are monomorphic or, if they are polymorphic, the polymorphism is disregarded (both alleles of a marker being scored as one). However, if the alleles of a polymorphic marker are distinguished and scored independently on the mapping panel, then haplotype information (i.e., the linkage phase between the alleles of two or more markers in the diploid genome) may be determined.

Haplotype information is of considerable interest, particularly in the human genome, where SNP haplotype information is valuable for a number of applications, including but not limited to the association between polymorphisms (particularly single-nucleotide polymorphisms, SNPs) and susceptibility to disease or to adverse reactions to drugs, which is currently being researched extensively; the association of SNP haplotypes with ‘normal’ variable traits across a population; and the use of SNP haplotypes to trace human population movements.

HAPPY mapping may be applied to haplotyping as follows. A mapping panel is prepared in the usual way, but marker detection and scoring is performed in such a way as to discriminate between the alleles of polymorphic markers (the two alleles of a marker are denoted here by upper- and lower-case, for example A, a), and the results for each allele recorded independently. If two or more polymorphic markers are thus scored, then the proximity between the alleles of each may be determined. For example, if the parental genome contains haplotypes AB and ab, and if marker A/a lies sufficiently close to marker B/b, then cosegregation (and hence linkage) will be observed between A & B, and between a & b, but not between A & b or between a & B. Hence, the parental haplotypes may be determined. The distance across which haplotype information can be obtained is determined by the size of the fragments used in preparing the mapping panel.

Many methods exist for the necessary scoring and discriminating between alleles, depending upon the nature of the polymorphism. Any of these methods may be applied in this context.

In many instances, the location of the markers in the genome will already have been determined, and only the linkage phase is required. In these cases, fewer panel members need to be analysed than would be necessary to determine the order and spacing of the markers a priori. Also, where map information already exists, the resolution of the mapping panel becomes almost irrelevant; hence, the panel can be prepared from DNA fragments as large as possible to maximise the range over which haplotypes may be determined. In an extreme case, the genomic DNA can remain unbroken, with complete chromosomal DNA molecules (or chromosomes) being segregated amongst the panel members; in this instance, the results will be unable to determine the order or spacing of markers along a chromosome, but will yield haplotype data over chromosomal distances. Ideally, HAPPY panels prepared for use in haplotyping should contain approximately twice as much DNA as those used for routine mapping, since each allele is considered as an independent marker. However, the acceptable range of DNA concentrations for standard HAPPY panels is broad enough to accommodate haplotyping.

The improved method of the present invention allows rapid haplotyping of a plurality of different markers in a single set of hybridisation reactions.

B. Mapping with Chromosomes

As mentioned above, it may be advantageous to map, or derive haplotype information, using whole chromosomes instead of broken nucleic acid. Moreover, the use of larger DNA fragments allows HAPPY mapping, normally useful at medium-to-high resolution, to be extended to provide low resolution maps.

In HAPPY mapping, DNA is conventionally first isolated in relatively pure form (in solution or in a protective gel matrix) before breakage by mechanical or other means. However, the fragile nature of long DNA molecules makes it difficult to manipulate fragments more than a few million basepairs (Mb) long. Hence, linkage between markers can only be easily determined over distances up to a few Mb.

The present invention describes several applications based upon the sampling and analysis (at limiting dilution) not of ‘naked’ DNA fragments but of complete chromosomes, fragments of chromosomes or chromatin. The natural packaging of DNA in these forms makes it possible to isolate and manipulate larger fragments than when handling naked DNA, including complete chromosomes.

DNA is released from cells in the form of either chromatin or metaphase chromosomes. In both of these forms, the DNA is stabilised and compacted by association with histones and other proteins (and may, optionally, be further stabilised by other treatments, such as the partial fixation techniques used when preparing metaphase chromosomes for in-situ hybridisation or flow-sorting). Mechanical breakage is then used to break the chromatin/chromosomes into fragments, a solution of which is diluted and dispensed into a panel of samples, each containing approximately 0.05-1.5 genome equivalents of DNA (but preferably similar amounts of DNA in each member of the panel); ranges between about 0.2 and about 1.5 are generally considered useful. The samples are then analysed in the way already described for the analysis of HAPPY mapping panels; pre-treatment with proteinase-K or other methods may in some cases be required to ensure that the DNA becomes amenable to PCR amplification.

Since intact metaphase chromosomes are routinely prepared and handled in solution, it is clear that the fragments which are segregated into the panel members can be of any size, up to and including complete chromosomes. (It will be noted that, once segregation into samples is complete, further fragmentation of the DNA is of no consequence as long as its marker content is preserved.) The distances over which linkage can best be detected by HAPPY mapping are typically up to 0.5-0.7 times the average length of the DNA fragments used. Hence, a panel made from coarsely-broken chromosome fragments can be used to make sparsely-populated maps in which the average distance between markers is several Mb or more. This is useful in those genomes whose size makes it impracticable or uneconomic to make the very dense maps produced by conventional HAPPY mapping.

Fragments of metaphase chromosomes can also be flow-sorted (indeed, fragmented chromosomes are normally seen as a ‘background’ when flow-sorting chromosomes, and arise through unwanted degradation or shearing of the desired intact chromosomes). Hence, flow sorting may be used (instead of dilution and random sampling) as a method to segregate the required number and sizes of fragments into the members of the mapping panel. Such an approach has the advantages that (a) the total amount of DNA in each panel member can be finely controlled and (b) the size range of fragments can be narrowly selected; such tight selection allows the range and resolution of the panel to be fine-tuned to address the mapping problem in hand, and improves the quality of the mapping data by excluding fragments which are either too small to reflect linkage between any markers, or are so large as to contain no useful mapping information.

Intact metaphase chromosomes may be segregated into the members of the mapping panel, either by limiting dilution of a solution of such chromosomes or by flow sorting. In this case, the panel will give no information on the order and spacing of markers within one chromosome, but will allow markers to be co-localised in groups to their respective chromosomes. This is of particular value for chromosomally assigning markers in those species in which the chromosomes cannot be distinguished by flow cytometry. For example, chromosomes 9, 10, 11 and 12 of human cannot be distinguished by flow cytometry; if a mapping panel were prepared by flow-sorting one or two chromosomes (sampled at random from the Chr9-12 cluster) into each panel member, then the typing of markers on the panel would quickly allow the markers to be assigned to chromosomal linkage groups.

All of the above methods may also be applied to determining haplotypes (the linkage phase between polymorphic loci). In such cases, it is necessary only to score the two alleles of each marker independently (using established techniques for discriminating between alleles); then, each allele can be treated as an independent marker, and (for example) linkage will be observed between A & B, and between a & b, revealing the haplotypes AB and ab. The use of chromatin/chromosome fragments or intact chromosomes in making the mapping panels allows haplotypes to be determined over considerable (or chromosomal) distances.

The invention is further described below, for the purposes of illustration only, in the following example.

### EXAMPLE

Nuclei are isolated from leaf cells of barley (Hordeum vulgare) and embedded in agarose strings. The strings are immersed in lysis solution (0.5 M EDTA, pH 9.0, 1% lauryl sarcosine sodium salt, 0.1 mg mL⁻¹ proteinase K) and incubated at 45° C. for 48 h with gentle mixing; then in 0.5 M EDTA, pH 9.0 for 1 hr at 45° C.; then in 0.05 M EDTA, pH 8.0 for 1 hr on ice; and stored in 0.05 M EDTA, pH 8.0 at 4° C. until needed. During this manipulation, the lysis solution diffuses into the agarose, lysing the nuclei and removing/degrading proteins and other nuclear material which diffuse out during the washing stages, leaving substantially pure DNA trapped within the agarose. The agarose serves to protect the DNA from unwanted mechanical breakage, since it is very important that the frequency of breaks in the DNA is controlled. Breaks are induced by gamma irradiation, sufficient to break the DNA into fragments of a wide range of sizes up to several megabases (Mb). Fragments of up to −2 Mb are run out by pulsed-field gel electrophoresis in low-melting-point agarose, alongside suitable size standards (chromosomal DNA isolated from Saccharomyces cerevisiae).

96 agarose plugs of constant, defined size are removed from a region of the gel containing barley DNA fragments of ˜1 Mb (as judged by the mobility of the known size standards) using a glass capillary. The amount of barley DNA loaded onto the gel, the duration of electrophoresis and the diameter of the capillary are selected such that ˜0.7 haploid equivalents of DNA fragments are expected in each agarose plug; the volume of each plug is <1 μl. Each plug is transferred to a separate well of a 96-well microtitre plate, supplemented with restriction enzyme buffer to a total volume of 5 μl, and overlaid with one drop (˜30 μl) of mineral oil to prevent evaporation. The samples are heated to 65° C. for 4 minutes to melt the agarose without denaturing the DNA, then cooled and maintained at 37° C.

A solution containing the restriction enzyme DpnII is added to each agarose plug to give a mixture containing 5 U of DpnII in a total volume of 7 μl of a buffer supporting digestion. The solution is incubated at 37° C. for 4 hr to allow cleavage of all DNA fragments at all sites recognised by the enzyme. The enzyme is then inactivated by heating to 65° C. for 45 min.

Oligodeoxynucleotides LIB1 (5′ AGT GGT ATT CCT GCT GTC AGG 3′) and LIB2 (5′ GAT CCC TGA CAG C 3′; the 3′ residue is a dideoxynucleotide to prevent extension of this oligonucleotide by DNA polymerase) are added to each well give a final concentration of 10 μM each oligo in a total volume of 12 μl. Oligo LIB1 carries a fluorescent moiety at its 5′ terminus. The solution is incubated at 65° C. for a further 20 min, and then cooled at 1° C. per minute to 15° C.

5 Units of T4 DNA ligase are added to each well, as is rATP to a final concentration of 1 mM in 15 μp total volume, and the reactions incubated at 14° C. for 15 hr. During this process, the 3′ end of LIB1 becomes ligated to the 5′ end of each strand of each restriction fragment, whilst LIB2 serves as a “splint” on the opposite strand; LIB2 cannot be ligated, as there is no available phosphate moiety at the 3′ end of the strands of the restriction fragments.

A solution of deoxyribonucleotide triphosphates (dATP, dCTP, dGTP, dTTP; collectively referred to herein as dNTPs) is added to each well, as is PCR buffer concentrate (Buffer 1 from the Expand Long PCR kit; Boehringer Mannheim) and 7 units of Expand Long DNA polymerase mix (Boehringer Mannheim) to give a final volume of 50 μl containing 400 μM of each deoxyribonucleotide triphosphate and 1× PCR buffer. The mixture is then thermocycled as follows: 14 cycles of 94° C.×40 sec, 57° C.×30 sec, 68° C. ×75 sec; then 34 cycles of 94° C.×40 sec, 57° C. ×30 sec, 68° C.×100 sec; then 1 cycle of 94° C.×40 sec, 57° C.×30 sec, 68° C.×5 min. During this cycling, priming is achieved by the surplus LIB1 oligo; LIB2 plays no further part in the reaction. This achieves global amplification of the majority of the linker-ligated restriction fragments, but preferentially of the smaller (<1 kb) fragments. The products of these amplifications are purified (i.e., unincorporated dNTPs and primers are removed) using commercially available kits and protocols.

A repertoire of markers is produced by cleaving barley genomic DNA with restriction enzyme DpnII, selecting fragments of <500 bp, and cloning these fragments in E. coli using the well-known pBluescript vector and established protocols. 20 recombinant clones are selected at random, and standard protocols are used to isolate recombinant plasmid DNA from each. This DNA is used to produce arrays comprising 20 markers by spotting onto glass slide substrates using a microarrayer. 96 replica slides are produced.

The mapping panel members are then hybridised to the probe arrays in a single series of hybridisation reactions, wherein the nucleic acids are incubated in an aqueous solution containing 6×SSC, 5× Denhardt's, 1% SDS (sodium dodecyl sulphate), 0.1 Na+ pyrophosphate and 0.1 mg/ml barley genomic DNA as competitor. Following hybridisation, high stringency washing is performed in several steps, with a final wash (about 30 min) at the hybridisation temperature in 0.2-0.1× SSC, 0.1% SDS. A suitable imaging system is used to identify those spots on each replica slide which display a signal indicative of hybridisation of the fluorescent amplified restriction fragments, and the results recorded.

The amplified fragments are then mapped relative to each other by seeing how often they co-segregate in the 96 aliquots. Occasionally, the marker cannot be mapped, either because the preamplification fails for any of various reasons or because the marker is not a single copy sequence (easily seen by noting the number of positives in the 96 aliquots) or because the sequence is a cloning artefact. The majority of markers, however, can be detected in a proportion of the samples and mapped by tabulation and calculation of linkage frequencies.

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A method for mapping a nucleic acid, comprising the steps of:
a) preparing a HAPPY mapping panel comprising a plurality of nucleic acid samples derived from a nucleic acid to be mapped, each member of the said panel containing a sampling of DNA fragments representing an amount equal by mass to 0.05 to 2 copies of the nucleic acid to be mapped;
b) labelling the nucleic acids in the mapping panel;
c) preparing a plurality of probes which are complementary to marker sequences present in the nucleic acid to be mapped and immobilising the probes on a solid phase;
d) bringing one or more members of the mapping panel into contact with each of said plurality of probes, such that the probes hybridise to sequences in the mapping panel which are complementary thereto;
e) removing non-hybridised nucleic acids and detecting hybridisation of mapping panel nucleic acids to the probe.

2. A method according to claim 1, wherein the panel members are preamplified prior to hybridisation with the probes.

3. A method according to claim 2, wherein the preamplification is non-specific.

4. A method according to claim 2 or claim 3, wherein the nucleic acids in the panel members are labelled during pre-amplification.

5. A method according to claim 1, wherein each of said one or more members of the mapping panel is screened with a plurality of probes simultaneously.

6. A method according to claim 5, wherein a plurality of members of the mapping panel is screened with a plurality of probes simultaneously.

7. A method according to claim 1, wherein the solid phase is a multiwell plate, a glass slide or a membrane.

8. A method according to claim 2, wherein the panel members are selectively preamplified to reduce mapping panel member complexity.

9. A method according to claim 8, wherein only sequences which comprise sequences complementary to probes are preamplified.


SEQUENCE LISTING (2 sequences)

SEQ ID NO: 1 - DNA, 21 bp, Artificial
  Oligonucleotide LIB1
agtggtattcctgctgtcagg

SEQ ID NO: 2 - DNA, 13 bp, Artificial
  Oligonucleotide LIB2
gatccctgacagc